(19) United States
(12) Patent Application Publication
(10) Pub. No.: US 2007/0092924 A1
(43) Pub. Date: Apr. 26, 2007

(54) Process for treatment of protein samples

(76) Inventor: Norman Leigh Anderson, Washington, DC (US)

Correspondence Address: Hendricks and Associates, P. O. Box 2509, Fairfax, VA, 22031-2509, US

(21) Appl. No.: 11/256,946

(22) Filed: Oct. 25, 2005

(51) Int. Cl. C12Q 1/37; C12P 21/06; C12M 3/00

(52) U.S. Cl. 435/23; 435/68.1; 435/287.1

(57) ABSTRACT

The process of the invention comprises 1) preparing a dry porous support incorporating one or more biomolecule-modifying reagents, 2) applying a biomolecule-containing sample solution to said support wherein the volume of said sample is equal to or less than the imbibition capacity of said support so that the sample is completely imbibed on said support, 3) allowing the product of step 2 sufficient time for said biomolecule-modifying reagent to interact with the biomolecule of interest in said sample, then 4) recovering biomolecules from said support and devices for use therewith

### FIELD AND BACKGROUND OF THE INVENTION

This invention relates to quantitative assays for evaluation of proteins in complex samples such as human plasma, and specifically to the preparation of samples for such assays. The invention can be used both for analysis of samples from a single individual source or, for purposes of evaluating the level of a particular protein in a population, can be used to analyze pooled samples from the target population.

There is a need for improvement in the collection and processing of human blood, plasma and serum for the measurement of disease-related proteins (i.e., use in clinical proteomics). The human plasma proteome holds the promise of a revolution in disease diagnosis and therapeutic monitoring, provided that major challenges in proteomics and related disciplines can be addressed. Plasma is not only the primary clinical specimen, but also represents the largest and deepest version of the human proteome present in any sample: in addition to the classical “plasma proteins”, it contains all tissue proteins (as leakage markers) plus very numerous distinct immunoglobulin sequences; and it has an extraordinary dynamic range, in that more than 10 orders of magnitude in concentration separate albumin and the rarest proteins now measured clinically. Abundant scientific evidence, from proteomics and other disciplines, suggests that among these are proteins whose abundances and structures change in ways indicative of many, if not most, human diseases. Nevertheless, only a handful of proteins are currently used in routine clinical diagnosis, and the rate of introduction of new protein tests approved by the US FDA has paradoxically declined over the last decade to less than one new protein diagnostic marker per year. Major advances in diagnostics are to be expected if certain technical problems in sample collection, preparation and analysis are solved. In this specification, I focus on issues of the collection and preparation of suitable samples from blood, although the disclosed processes can be used for other sample types as well.

Human blood, and the serum and plasma samples derived from it, is typically collected and prepared in evacuated glass or plastic tubes. In the usual course of medical practice, these tubes are filled by venipuncture and sent to a clinical laboratory for analysis, where they may be stored for extended periods (hours to days) at room temperature or 4 C. It would be useful for purposes of clinical proteomics to handle blood samples in such a way that the cells are removed and the remaining plasma (or serum) proteins stabilized against chemical change much more rapidly than this. Ultimately it would be useful to carry out such preparations on small amounts of blood obtainable by skin prick instead of venipuncture, thus allowing collection of plasma samples for protein measurement by a patient at home.

For those analytical procedures that require digestion of the plasma or serum to peptides (e.g., by exposure to a proteolytic enzyme such as trypsin), it would be useful to provide a means for executing this digestion conveniently and reproducibly on freshly acquired samples without a requirement for expensive automation or preparation of fresh reagents.

The present invention addresses these problems by providing means for blood collection, preparation of plasma, denaturation of the plasma proteins, digestion of the plasma proteins to peptides, and stabilized storage of the peptides in a process performed by a simple single use device. Additional reagents, such as synthetic stable-isotope labeled peptides used as internal standards for quantitation, can be incorporated as well. The invention is equally applicable to protein samples from other sources, such as tissue homogenates, animal, plant or microbial samples, other body fluids, environmental samples and the like. Other biomolecules, such as DNA and RNA, can likewise be collected, processed and stabilized.

One important advance that can help expand the diagnostically useful proteome is the use of many protein measurements together as a panel, so that patterns of change can be associated with disease or treatment, instead of relying on single protein markers interpreted alone. Several streams of scientific effort have generated data supporting this approach. More than 20 years ago it became clear that different tumor cell types could be distinguished based on patterns of metabolites analyzed by GC-MS (Jellum, Bjornson, Nesbakken, Johansson, and Wold, J Chromatogr 217:231-7, 1981), and that a panel of biochemical markers (most of them simple low molecular weight clinical analytes) could “recognize” individuals within a group over periods of years (Robertson, Van Steirteghem, Byrkit, and Young, Clin Chem 26:30-6, 1980) when analyzed by appropriate multivariate statistical methods. There were efforts to use the latter approach to detect disease signatures in then-standard 20-analyte serum chemistry panels, but these met with little success, probably due to the character and small number of the analytes. Quantitative serum protein proteomics was used to show that a panel of high-abundance acute phase-related proteins could give a better statistical measure of inflammation than the classical marker serum amyloid A (Doherty, Littman, Reilly, Swindell, Buss, and Anderson, Electrophoresis 19:355-63, 1998). Cancer tissue samples could be analyzed to distinguish tumor type and prognosis (Schmid, Schmitter, Blum, Miller, and Vonderschmitt, Electrophoresis 16:1961-8, 1995) and a panel of six cancer markers in plasma was found useful (Negishi, Furukawa, Oka, Sakamoto, Hirata, Okabe, Matayoshi, Akiya, and Soma, Gynecol Obstet Invest 23:200-7, 1987; Alaiya, Franzen, Hagman, Silfversward, Moberger, Linder, and Auer, Int J Cancer 86:731-6, 2000). More recently, mass spectrometry-based proteomics approaches have been used as well to discover patterns of disease-related protein features related to a specific cancer (Petricoin, Ardekani, Hitt, Levine, Fusaro, Steinberg, Mills, Simone, Fishman, Kohn, and Liotta, Lancet 359:572-7, 2002).

Whole proteins can be analyzed by an approach termed SELDI-TOF (for surface-enhanced laser desorption ionization-time of flight) mass spectrometry, a variant of MALDI-TOF (matrix-enhanced laser desorption ionization-time of flight), in which chemical fractionation based on protein affinity for derivatized MS targets is used to reduce sample complexity to a level at which whole-protein MS can resolve a series of individual peaks. This approach has been implemented commercially (e.g., by Ciphergen and Lumicyte) and used to discover protein peaks that may be associated with disease (e.g., the putative ovarian cancer markers discovered by Petricoin et al (Petricoin, Ardekani, Hitt, Levine, Fusaro, Steinberg, Mills, Simone, Fishman, Kohn, and Liotta, Lancet 359:572-7, 2002)). A significant disadvantage of the approach is that MS analysis of whole proteins does not directly provide a sequence-based identification (there being many proteins with close to a given mass), and hence the protein peaks discovered as markers are not strictly speaking identified without significant additional effort. In particular, without a discrete identification, it is not generally possible to demonstrate that a peak is one protein analyte, nor to translate the measurement into a classical immunoassay format.

A more general approach involves digesting proteins (e.g., with trypsin) into peptides that can be further fragmented (MS/MS) in a mass spectrometer to generate a sequence-based identification. The approach can be used with either electrospray (ESI) or MALDI ionization, and is typically applied after one or more dimensions of chromatographic fractionation to reduce the complexity of peptides introduced into the MS at any given instant. Optimized systems of multidimensional chromatography, ionization, mass spectrometry and data analysis (e.g., the multidimensional protein identification technology, or “MudPIT” approach of Yates, also referred to as shotgun proteomics) have been shown to be capable of detecting and identifying ˜1,500 yeast proteins in one analysis (Washburn, Wolters, and Yates, Nat Biotechnol 19:242-7, 2001), while a single dimensional LC separation, combined with the extremely high resolution of a Fourier-transform ion cyclotron resonance (FTICR) MS identified more than 1,900 protein products of distinct open reading frames (i.e., predicted proteins) in a bacterium (Lipton, Pasa-Tolic', Anderson, Anderson, Auberry, Battista, Daly, Fredrickson, Hixson, Kostandarithes, Masselon, Markillie, Moore, Romine, Shen, Stritmatter, Tolic', Udseth, Venkateswaran, Wong, Zhao, and Smith, Proc Natl Acad Sci U S A 99:11049-54, 2002). In human urine, a sample much more like plasma than the microbial samples mentioned above, Patterson used a single LC separation ahead of ESI-MS/MS to detect 751 sequences derived from 124 different gene products. Very recently, Adkins et al have used two chromatographic separations with MS to identify a total of 490 different proteins in human serum (Adkins, Varnum, Auberry, Moore, Angell, Smith, Springer and Pounds, Mol Cell Proteomics 1:947-55, 2002), thus substantially expanding the proteome. Such methods should have the ability to deal with the numerous post-translational modifications characteristic of many proteins in plasma, as demonstrated by its ability to characterize the very complex post-translational modifications occurring in aging human lens (MacCoss, McDonald, Saraf, Sadygov, Clark, Tasto, Gould, Wolters, Washburn, Weiss, Clark, and Yates, Proc Natl Acad Sci U S A 99:7900-5, 2002) (73 modified sites characterized in 11 crystallins). The sensitivity obtainable in such an analysis has been tested by Wu et al (Wu, Amato, Biringer, Choudhary, Shieh, and Hancock, Journal of Proteome Research in press) (Wu, Amato, Biringer, Choudhary, Shieh and Hancock, J Proteome Res 1:459-65, 2002) using human growth hormone spiked into human plasma at a concentration of 16 ng/ml. Using only a reverse phase separation to resolve a tryptic digest of whole spiked plasma, a single human growth hormone (hGH) peptide was observed (among 200+ proteins apparently identified), and with additional fractionation processes, additional hGH peptides were seen, confirming its detection.

Preparation of peptides from a sample such as plasma is typically carried out by first denaturing the protein sample (e.g., with sodium dodecyl sulfate (SDS), organic solvents, urea or guanidine HCl), reducing the disulfide bonds in the proteins (e.g., with dithiothreitol or mercaptoethanol), alkylating the cysteines (e.g., by addition of iodoacetamide which reacts with the free —SH group of cysteine), quenching excess iodoacetamide by addition of more dithiothreitol or mercaptoethanol, and finally (after removal or dilution of the denaturant) addition of the selected proteolytic enzyme (e.g. trypsin), followed by incubation to allow digestion. Following incubation, the action of trypsin is terminated, either by addition of a chemical inhibitor (e.g., diisopropylfluorophosphate (DFP) or phenylmethylsulfonylfluoride (PMSF)) or by denaturation (through heat or addition of denaturants, or both) or removal (if the trypsin is on a solid support) of the trypsin. Most commonly, these steps are carried out in solution by addition of reagents and, where necessary, by exchange of solvents. Finally the peptides generated by digestion can be trapped on, and later recovered intact from, a suitable solid adsorbent (such as a C18 reverse phase matrix).

Numerous alternatives have been explored for each step in the above general procedure. Of particular interest is the introduction of tributyl phosphine (TBP) and tris(2-carboxyethyl)phosphine (TCEP) as reducing agents for cystine disulfides (e.g., see Getz, E. B., M. Xiao, et al. (1999) Anal Biochem 273(1): 73-80). These reagents can be used together with denaturants and a thiol-alkylating agent under certain circumstances, thus providing the possibility of a single step denaturation/reduction/alkylation process.

Recent Lee, et al, have shown that enzymatic cleavage (e.g., by trypsin) can be made more rapid through the use of very high concentrations of trypsin immobilized on a membrane support (Li, Cooper and Lee, J Chromatogr A 979:241-7, 2002, Cooper, Chen, Li and Lee, Anal Chem 75:1067-74, 2003). They used Millipore's Immobilon Psq polyvinylidine difluoride (PVDF) membrane as a solid support onto which trypsin would spontaneously adsorb from a 2 mg/ml solution, and on which the adsorbed trypsin showed 85% of its solution activity towards a protein mixture pumped through the membrane. Increased temperatures also promote more rapid and complete digestion (Venkatesh, R. and P. V. Sundaram (1998) Ann N Y Acad Sci 864:512-6.).

Trypsin has also been immobilized on various beaded supports, and is offered as a commercial product immobilized on Poros™ media (the Porozyme™ material sold by Applied Biosystems, Inc), on agarose media (e.g., Sigma Aldrich product T1763), or on PVDF (Bienvenut, Sanchez, Karmime, Rouge, Rose, Binz and Hochstrasser, Anal Chem 71:4800-7, 1999; Binz, Muller, Hoogland, Zimmermann, Pasquarello, Corthals, Sanchez, Hochstrasser and Appel, Curr Opin Biotechnol 15:17-23, 2004) (Bienvenut and Hochstrasser, U.S. Pat. Nos. 6,221,626 and 6,632,339). In each of these cases, the immobilized trypsin is used to digest proteins or peptides flowing through the support, driven through the support by electrophoresis or diffusing into it from an adjacent external volume.

In the descriptions that follow, quantitation of proteins, peptides and other biomolecules is addressed in a general sense, and hence the invention disclosed is in no way limited to the analysis of plasma and other body fluids. The instant invention uses several of the cited methods of the prior art in an entirely different combination.

### SUMMARY OF THE INVENTION

The present invention relates to processes for processing biomolecule-containing samples, including, for example, the collection and denaturation of plasma proteins, and the digestion of proteins to peptides. The invention provides a means for exposing a sample of biomolecules in solution to a fixed relative concentration of biomolecule-modifying reagent (e.g., a denaturant or an enzyme), in a convenient form for manual or automated sample processing. In the invention, a biomolecule-containing liquid sample is imbibed into a porous support (typically a membrane, foam or porous bead) loaded with a known amount of one or more biomolecule-modifying reagent: the support's included volume defines the fluid volume imbibed, and the reagent load defines the relative reagent concentration. Thus as long as the membrane has sufficient volume to completely imbibe an applied sample volume, the ratio of reagent to sample volume is uniform and fixed for the whole sample. Certain important advantages arise in specific applications. For example, certain supports are capable of binding a high loading of protease, thus making it possible to raise the protease-to-protein substrate ratio from the conventional 1:80 to a value close to 1:1. This improved ratio allows faster sample processing and more complete digestion, the latter being critical for quantitative applications, and the sample-derived peptides are substantially free of contamination by protease or protease fragments in solution. Similarly a protein denaturant can be imbibed into a membrane at the desired final working concentration and dried in place, so that when a protein sample is later imbibed into the same volume of membrane, the final denaturant concentration achieved upon dissolution of dry denaturant into the sample is the desired final working concentration. In either of these illustrative cases (digestion or denaturation) the resulting biomolecules (in this case proteins or peptides) can be removed from the support's included volume by filtration (i.e., by passage of wash fluid through the membrane, either laterally through the membrane or normal to the membrane) or by diffusion into another fluid volume.

The present invention further provides processes useful with devices that execute one or a combination of the following steps on protein-containing samples: 1) separation of dissolved proteins from cells and cell debris; 2) addition of internal standards for quantitation; 3) denaturation of proteins; 4) reduction of cystine disulfide bonds in proteins; 5) alkylation of cysteine thiols; 6) digestion of proteins to peptides; 7) capture and purification of peptides. The devices disclosed carry out one or more of these steps without the addition of liquids other than a protein-containing sample and a simple buffer, with the remaining reagents being packaged within the device. Many capabilities of the invention derive from the use of materials developed for lateral flow diagnostic devices in a new context with new reagents and chemical processes. Materials such as blood filter pads, conjugate release pads, imbibing membranes, protein adsorbing membranes and wicking pads are manufactured by a number of companies (including Millipore, Schleicher & Schuell, Whatman, and Sartorius) for inclusion in such diagnostic devices.

The process of the invention comprises 1) preparing a dry porous support incorporating one or more biomolecule-modifying reagents, 2) applying a biomolecule-containing sample solution to said support wherein the volume of said sample is equal to or less than the imbibition capacity of said support so that the sample is completely imbibed on said support, 3) allowing the product of step 2 sufficient time for said biomolecule-modifying reagent to interact with the biomolecule of interest in said sample, then 4) recovering biomolecules from said support and devices for use therewith

Some of the devices for use in the processes of the invention may be used for obtaining and retaining samples of proteins in supports wherein the proteins are free from cellular materials so that the cell-free proteins can be stored and shipped easily.

### BRIEF DESCRIPTION OF THE DRAWINGS

FIG 1. Imbibition of a sample into a porous support mounted as the filter of a 96-well filter plate.

FIG. 2. Removal of a sample from the porous support of FIG. 1 by vacuum filtration.

FIG. 3. A well containing multiple layers of porous support.

FIG. 4. Imbibition of sample into a sheet of porous support.

FIG. 5. Exposure of sample proteins to a dissolvable reagent dried in a porous support.

FIG. 6. Use of a two-layer support to improve spreading of sample.

FIG. 7. Use of a two-zone support, in which the first zone provides a near-saturating load of reagent.

FIG. 8. Use of multiple timed-release reagents to effect multi-step chemistry.

FIG. 9. Use of a blood filter to remove cells from whole blood applied to a support containing a protein modifying agent.

### DETAILED DESCRIPTION OF THE INVENTION

The invention provides processes and associated devices for treating protein-containing samples, and particularly for effecting treatments useful in preparing those samples for identification and quantitative analysis of peptides and/or proteins therein. While many of the methods known in the art and disclosed above are useftil with the methods of the invention, the process for such a commercially useful process has not previously been disclosed.

The terms “analyte”, and “ligand” may be any of a variety of different molecules, or components, pieces, fragments or sections of different molecules that one desires to measure or quantitate in a sample. The term “monitor fragment” may mean any piece of an analyte up to and including the whole analyte which can be produced by a reproducible fragmentation process (or without a fragmentation if the monitor fragment is the whole analyte) and whose abundance or concentration can be used as a surrogate for the abundance or concentration of the analyte. The term “monitor peptide” means a peptide chosen as a monitor fragment of a protein or peptide.

The term “biomolecules” refers to any molecule present in a biological system, and includes proteins, nucleic acids (specifically DNA and RNA in its various forms, both intracellular and extracellular), complex sugars (glycans and the like), lipids, and a variety of metabolites.

The term “biomolecules-modifying reagent” refers to any chemical (including enzymes) that effects a modification in the covalent or non-covalent structure of a biomolecule.

The term “protein-modifying reagent” refers to any of several classes of molecules which, when applied to a sample containing proteins, cause one or more changes in the structure of at least one protein present in a sample. Changes in structure include covalent modifications (e.g., cleavage of the protein into peptides, removal of sugar structures from glycoproteins, reduction and alkylation of cystine sulfhydrals, etc.) and non-covalent modifications (e.g., unfolding of 3-D structure, dissociation of protein complexes, etc.). Protein-modifying reagents can be immobilized (i.e., fixed to a support), located in a support but dissoluble, or in solution.

The terms “proteolytic treatment” or “enzyme” may refer any of a large number of different enzymes, including trypsin, chymotrypsin, lys-C, v8 and the like, as well as chemicals, such as cyanogen bromide. In this context, a proteolytic treatment acts to cleave peptide bonds in a protein or peptide in a sequence-specific manner, generating a collection of peptide fragments referred to as a digest.

The term “denaturant” includes a range of chaotropic and other chemical agents that act to disrupt or loosen the 3-D structure of proteins without breaking covalent bonds, thereby rendering them more susceptible to proteolytic treatment, more soluble, or both. Examples include chaotropes such as urea, guanidine hydrochloride, ammonium thiocyanate; detergents such as sodium dodecyl sulfate, cetyltrimethyl ammonium bromide, Triton X-100; as well as solvents such as acetonitrile, ethanol, methanol and the like.

The term “reverse-phase matrix” and “C18” are meant to include any of a variety of hydrophobic surface phases (such as C18 or C8 aliphatic hydrocarbons) presented on the surface of a solid support and in contact with aqueous solvent.

The term “bind” includes any physical attachment or close association, which may be permanent or temporary. Generally, reversible binding includes aspects of charge interactions, hydrogen bonding, hydrophobic forces, van der Waals forces, etc., that facilitate physical attachment between the molecule of interest and the analyte being measured. The “binding” interaction may be brief as in the situation where binding causes a chemical reaction to occur. Reactions resulting from contact between the binding agent and the analyte are also within the definition of binding for the purposes of the present invention, provided they can be later reversed.

The terms “internal standard”, “isotope-labeled monitor fragment”, or “isotope-labeled monitor peptide” may be any altered version of the respective monitor fragment or monitor peptide that is 1) recognized as equivalent to the monitor fragment or monitor peptide by the appropriate binding agent and 2) differs from it in a manner that can be distinguished by a mass spectrometer, either through direct measurement of molecular mass or through mass measurement of fragments (e.g., through MS/MS analysis), or by another equivalent means.

“SIS” or “stable isotope standard” means a peptide having a unique sequence derived from the protein product of a single gene and including a label of some kind (e.g., a stable isotope) that allows its use as an internal standard for quantitation (see U.S. patent application Ser. No. 10/676,005 “High Sensitivity Quantitation of Peptides by Mass Spectrometry”). Included peptides may have non-material modifications of this sequence, such as a single amino acid substitution (as may occur in natural genetic polymorphisms), substitutions outside the region of contact (including covalent conjugations of cysteine or other specific residues), or chemical modifications to the peptide (including glycosylation, phosphorylation, and other well-known post-translational modifications) that do not materially affect binding.

The term “support” includes any porous material in membrane, sheet, bead, plug, particulate or other forms whose structure defines an included volume, and which can imbibe a liquid sample by capillary action or surface tension. A support can consist of one or more porous materials embedded or dispersed within other porous materials. A support can also be composed of particles embedded within another porous material (e.g., 3M Empore® membranes). The material of the support, and particularly the surface (internal and external) exposed to an imbibed liquid, is referred to as the matrix.

The term “imbibition” means the absorption of liquid into a porous support without pressure by means of capillary forces, and applies to supports that swell as well as those that do not.

“Immobilized enzyme”, means any form of enzyme that is fixed to the matrix of a support by covalent or non-covalent interaction such that the majority of the enzyme remains attache to the support of the membrane.

By the term “imbibe” is meant to describe the process whereby a liquid is drawn into a porous material by forces of capillary action or surface tension. When a liquid sample is fully imbibed into a support, it is fully contained within the support, leaving minimal residual liquid outside the volume described by the outer surface of the support. The process of imbibition into a homogeneous support zone ensures that all elements of the liquid are exposed equally to enzymes or reagents evenly distributed within the support zone.

The process of the invention requires the following steps: 1) preparation of a porous support incorporating one or more biomolecule-modifying reagents in the support, 2) application of volume of sample solution which is equal to or less than the imbibition capacity of the support, 3) allowance of sufficient time for the sample imbibed in the support to interact with biomolecule-modifying reagents in the support, then 4) removal of biomolecules from the support. The retention time of step 3 can be extended, providing methods for storing biomolecules in stable form. It may, be possible to dry the product of step 3 in order to store biomolecules. The temperatures required for appropriate interaction of sample and reagents will depend on the particular biomolecules of the sample and reagents.

In one set of embodiments, a porous support in the form of a membrane containing an immobilized proteolytic enzyme such as trypsin is used to digest an imbibed protein sample. The support may consist of conventional porous membranes such as PVDF, nitrocellulose, nylon, glass fiber, etc., and can bind proteins directly or can be activated so as to bind an adsorbed protein covalently. A second group of embodiments employ the invention to process a blood or plasma sample so as to produce a stable, denatured protein sample suitable for storage or shipment.

In one embodiment, a PVDF membrane incorporated as the filter element in a multiwell filter plate is used as the support. Each well of such a plate has a disk of PVDF membrane serving as the well-bottom, supported in such a way that liquid placed in the well can be drawn through the membrane by vacuum applied beneath the membrane or by centrifugal force, and the flow-through liquid collected in a 96-well receiver plate beneath the filterplate.

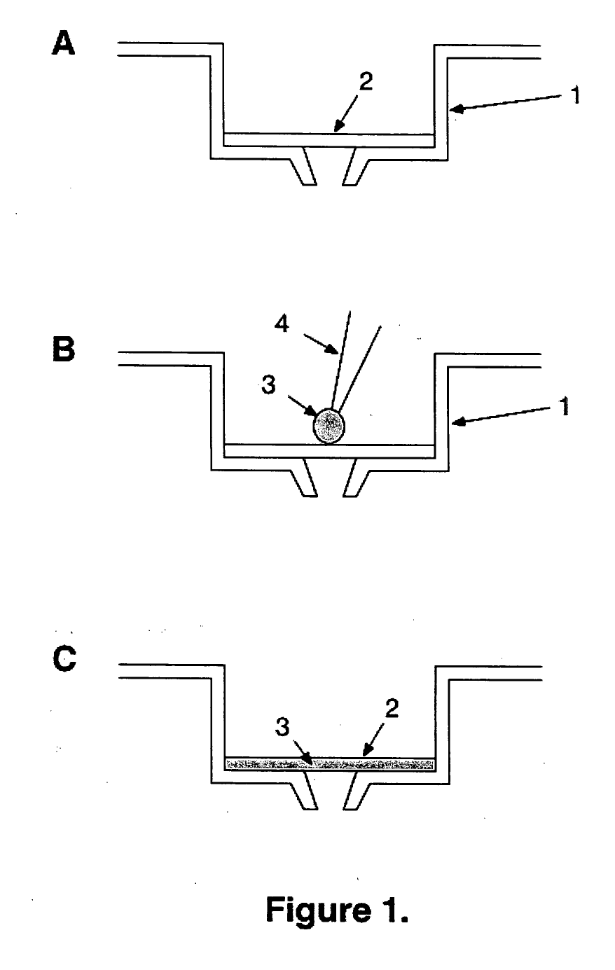

FIG. 1A shows a cross-sectional view of one well of a 96-well filter plate, showing a wall 1 having an exit opening at its lowest point and filter membrane 2 which forms a porous filter over the entire bottom of the well, including the exit opening. In FIG. 1B a volume of sample 3 is placed on the filter membrane by pipette 4. In FIG. 1C sample 3 is completely imbibed into filter membrane 2, exposing its protein contents to a uniform concentration of enzyme immobilized on membrane 2 during an incubation step.

The membrane is loaded with protease by treating it (after initial wetting with methanol and rinsing with aqueous buffer, in the case of PVDF) with a concentrated solution (e.g., 2 mg/ml) of a biomolecule-modifying agent (such as trypsin), followed by removal of excess unadsorbed enzyme through washing with ammonium bicarbonate, another volatile buffer, or with a dilute acidic buffer. Finally, the membrane is dried, either simply by exposure to the air or by drawing air through the membrane via an applied vacuum. The matrix of the porous membrane is thus coated with the enzyme, yielding a very high local enzyme concentration. If Millipore Immobilon-P (a 0.45 micron PVDF membrane) is used (available commercially in 96-well filterplates as Millipore MSIPN4510 96-well MultiScreenHTS Plates with hydrophobic Immobilon-P PVDF membrane) then approximately 96 ug/cm2 of a typical protein can be bound, or 35 ug/well given a membrane area of 0.37 cm2 in each well. Up to three times as much protein can be bound if 0.2 micron Immobilon PSQ is substituted for Immobilon-P.

The membrane can then be dried, rendering it capable of imbibing a volume of sample solution to be digested. Enzyme activity can be rendered more stable to drying and rehydration in sample by incorporation of protective agents in the membrane at the time of drying (including sucrose, trehalose, or other protectants known in the art).

Leaching of trypsin off the membrane during digestion introduces trypsin and its autolysis peptides as contaminants of the processed protein sample. Such contamination is generally undesirable and can be reduced by optional cross-linking of the trypsin molecules together after they are immobilized on the membrane. Well known bifunctional covalent crosslinking agents such as dimethylpimelimidate can be used directly, or agents like glutaraldehyde used together with a subsequent reduction step (e.g., with sodium borohydride). By joining most trypsin molecules together with their neighbors, the possibility that they can come off the membrane is reduced.

Another alternative method of preventing reagent leaching during digestion is use of covalent chemical coupling in the initial trypsin binding step. Covalent binding of enzyme to the membrane is advantageous in preventing leakage of the enzyme into an imbibed sample (thereby contaminating the resulting digest). Covalent immobilization can be achieved by use of a pre-activated membrane (e.g., Sartorius Sartobind® epoxy-activated membrane) or a PVDF membrane, which has been chemically derivatized to introduce protein reactive groups (as described by Bienvenut and Hochstrasser in U.S. Pat. Nos. 6,221,626 and 6,632,339) in place of conventional hydrophobic PVDF. A further alternative is the use of a membrane which incorporates particles capable of covalently binding protease, e.g., the Teflon-entrapped swellable azlactone-functionalized particles found in 3M Empore® AZ membranes (3M Corporation, St. Paul, Minn.). The latter beads swell during binding (through uptake of aqueous solvent), and, after coupling and washing according the manufacturer's instructions, the filters are dried. In this case the membrane shape is distorted by the swelling of the particles, and does not return to a flat shape upon drying. After drying, an applied sample (˜60 ul/well) reswells the beads with plasma proteins (˜300 ug), which are then digested by a protease such as trypsin (of which ˜200 ug/well can be covalently bound), yielding a trypsin:substrate ratio of ˜1:1.5. By applying a smaller volume of sample than the maximum swollen volume of the protease-loaded beads (e.g., 50 ul), optionally followed by small volume of buffer (e.g., 10 ul) to complete swelling, complete imbibition of sample proteins can be achieved. Peptides are subsequently recovered by flow-through of an added volume of wash liquid (not imbibed), as for conventional membrane filter plates.

As an examples, the possibility of trypsin autolysis during sample digestion can be further reduced by optional reductive alkylation of the immobilized enzyme: in this case after enzyme immobilization the membranes can be successively exposed to solutions of formaldehyde, followed by sodium borohydride (or cyanoborohydride) and finally a wash of ammonium bicarbonate. This process converts the trypsin's lysines to dimethyllysine, which is a poor substrate for tryptic activity thus reducing the chances of generating trypsin autolysis peptides.

For some membranes, such as nitrocellulose, the membrane, now coated with a hydrophilic coating of enzyme (and optionally with protective agent such as sucrose), easily wets with an aqueous protein solution such as serum or plasma. For less hydrophilic membranes such as a hydrophobic PVDF, even a coating of protein (e.g., trypsin) does not render the dried membrane directly wetable by plasma or plasma diluted by addition of conventional denaturants such as urea or GuHCl or typical buffers. However, an aqueous solution containing a moderate concentration (e.g., 20-40% in water) of an agent such as propylene glycol (PG) will wet protease-coated PVDF. Thus the addition of PG to a protein sample before application to a trypsin-loaded PVDF membrane makes possible the up-take by the membrane by imbibition, achieving the goal of this invention. Other volatile wetting agents such as ethanol, methanol, and isopropanol may also be added to achieve wetting and sample imbibition. Certain detergents compatible with subsequent analytical processes can also be used. These include low concentrations of sodium dodecyl sulfate, or higher concentrations of chemically cleavable detergents such as Millipore's RapiGest® product.

In order to facilitate addition of monitor peptide standards (SIS) at know concentrations into the final sample digest, the SIS can optionally be applied to the membrane and dried in place, so as to dissolve in the sample added later. In this case, addition of a defined sample volume chosen to fully occupy the membrane is important to preserve the desired known standard concentration.

The treated membrane is used to digest a protein sample (with suitable wetting properties) applied to its surface. The volume of sample is chosen so as to be less than or equal to the membrane's included volume (˜6.5 ul for a 200 micron thick PVDF membrane of 0.68 cm diameter, as supplied in typical filter plates). Thus the applied sample is entirely imbibed into the membrane, where every element of volume contains the same high concentration of immobilized trypsin.

Plasma samples are prepared for digestion by denaturation, reduction and alkylation. The purpose of such treatments is to open up the compact structures of the proteins, dissociate protein complexes and render each appropriate cleavage site (in the case of trypsin most lysines and arginines in the protein sequence) available to the proteolytic enzyme for cleavage. Since cystine intra- or inter-chain disulfide bonds play a major role in inhibiting protein unfolding, the reduction of cystine to two cysteines residues, and the modification of the resulting cysteines so as to prevent reformation of cystine bridges, are desirable steps in the sample preparation process. Briefly, each sample is subjected to dissociation (e.g., by addition of two volumes of 9M urea or 8M GuHCl), followed by cystine disulfide reduction (by addition, in minimal volumes, of dithiothreitol or mercaptoethanol to a concentration of 2-4× the concentration of sample cysteine thiols, estimated at 26 mM in plasma before dilution), and, after incubation for 30 min at 60° C., alkylation of cysteines (by addition, in minimal volume, of iodoacetamide, iodoacetic acid, or the like, to a concentration 2× that of DTT just added) and incubation in the dark. Finally, samples are diluted so as to achieve a denaturant concentration at which the proteolytic enzyme on the membrane can be active (e.g., to <2M urea or 1M GuHCl) by addition of 11 volumes (based on original plasma sample volume) of 100 mM ammonium bicarbonate (AMBIC) buffer, pH 8.5 in 50% propylene glycol, 50% water. These steps result in a plasma dilution of approximately 16-fold, and giving a final protein concentration of about 5 mg/mi.

Given the typical 200 micron thickness of PVDF membranes and the typical geometry of 96-well filter plates (based on microtiter plate specifications), the included volume of the membrane in one well is roughly 6 ul. Thus 6 ul of prepared plasma sample (equivalent to ˜0.6 ul of the original plasma, and containing ˜30 ug total protein) is delivered onto the surface of the membrane at the bottom of the well, where it is imbibed by the PVDF membrane forming the well bottom. For example, since 0.45 micron PVDF can bind ˜100 ug trypsin per cm2, and the well bottom is about 0.33 cm2 in area, the filter can contain ˜30 ug of immobilized trypsin. Thus the ratio of trypsin to plasma protein can be on the order of 1:1. Lower loadings of trypsin can be used if required.

Membrane-immobilized proteolytic enzyme, present at high concentration relative to total sample protein in the prepared plasma sample, rapidly cleaves the sample proteins. At a point in time when the cleavage is judged to be optimal, the membrane can be frozen (thus suspending the cleavage reaction), or if a volatile solvent is used, the membrane can be dried. Drying of the sample-loaded membrane easily provides a storable, digested sample in a form from which peptides can later be extracted for analysis. The advantage of such a storage form is the prevention of further modification of sample proteins by the action of sample enzymes (because no active sample-derived enzymes should remain: the sample peptides are too small to carry out enzymatic modifications on one another).

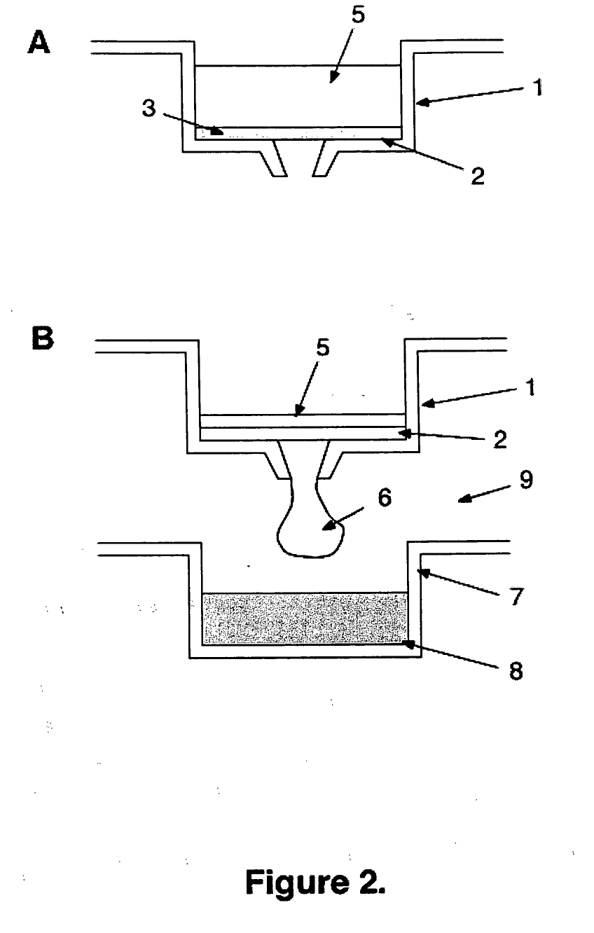

Alternatively the sample-derived peptides can be recovered at this point by diffusion into an added volume of recovery solvent, or the peptides can be removed from the membrane by flowing fluid through it and collecting the peptides in this fluid for further processing and analysis (the preferred approach shown in FIG. 2).

In FIG. 2A, a volume of wash solution 5 is placed in well 1 above filter 2 containing imbibed sample 3. In FIG. 2B, the plate containing well 1 is mounted over a receiver plate 7 in a vacuum filtration device capable of creating a relative vacuum in gap 9, such that wash solution 5 is drawn through filter 2, exiting through the exit hole as droplets 6 which fall into the receiver plate. The contents of the receiver plate well 8 comprise the sample-derived products 3 diluted by the wash buffer 5.

Thus to recover digest peptides by flow-through, the filterplate is placed over a receiver plate in a vacuum filtration device designed for 96-well plates, and a suitable volume (˜200 ul) of buffer (e.g., 100 mM ammonium bicarbonate) is applied to each well. The buffer is then drawn through the PVDF by applied vacuum, and the flow-through liquid, containing the peptides, is collected in the receiver plate. In the preferred case, the receiver plate is a 3M Empore C18 filter plate which has previously been wetted with methanol and equilibrated with 100 mM ammonium bicarbonate buffer by washing. The vacuum device is then reassembled with the Empore filterplate as the top component, and the peptide solution drawn through it by vacuum, with the flowthrough either collected or else discarded (since it is presumed to be depleted of peptides, which are captured on the C18 material of the Empore plate). Finally, the Empore plate is washed with additional buffer and placed over a fresh receiver plate, while a peptide-eluting solution (50% acetonitrile, 0.1% TFA in water) is applied to each well and vacuum used to draw this solution through the C18 bed, eluting the peptides into the final receiver plate. This final plate is then subjected to evaporation in a SpeedVac so as to dry the peptides down, after which they are redissolved in an aqueous solvent for analysis, typically on an LC-MS/MS analytical platform whose LC separation is a reversed phase (C18) separation.

Centrifugal means, using rotors designed to carry a filterplate and (below it) a receiver plate, are also suitable for forcing recovery solvent through the membrane in order to recover sample biomolecules.

It will be evident to those skilled in the art that the PVDF filterplate membrane serves the purpose of exposing a fixed volume (determined by the inbibition capacity of the membrane) of plasma sample to a fixed amount of reagent, at a high relative reagent:plasma protein ratio, but under circumstances such that the reagent does not appear in the final sample at this high relative abundance. The digestion is accomplished by simple addition of sample to the membrane, and thus does not require handling of reagent (which is immobilized on the plate during a previous manufacturing step). The digestion process is rapid, and the plates can in principle be re-used. Likewise it will be evident to those skilled in the art that other sample processing steps can be carried out by protein modifying agents immobilized on a porous support and used in the manner described. These alternatives include cleavage with other proteolytic enzymes, enzymes that remove or modify sugar moieties on proteins (e.g., a neuraminidase acting on glycoproteins to remove sialic acid), phosphatases that remove phosphoprotein phosphate groups, and non-protein alternatives including immobilized protein cleavage reagents.

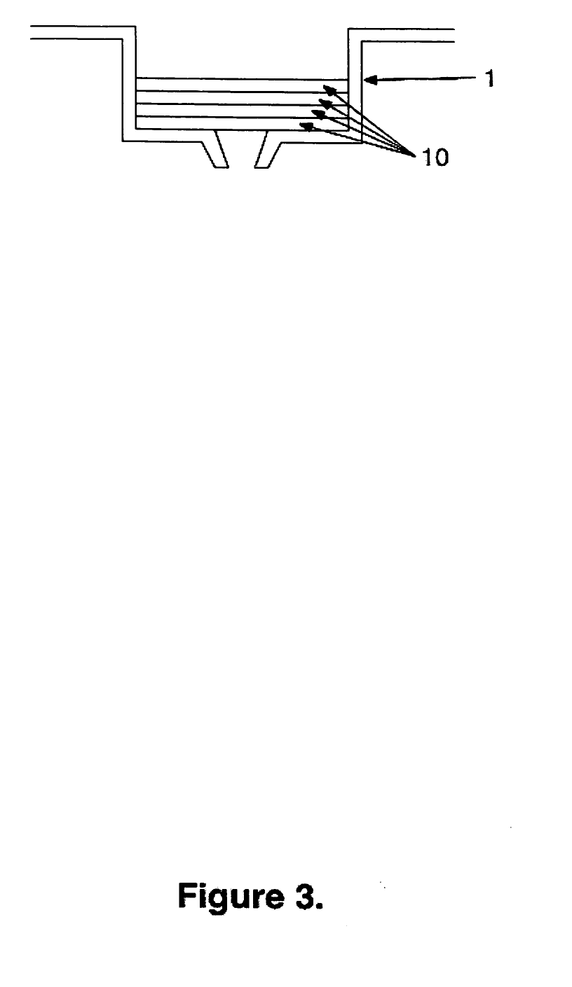

In another preferred embodiment, a filterplate is used that has multiple layers of PVDF stacked at the bottom, providing increased volume for sample. When 10 layers are provided, the membrane volume is increased to 60 ul, allowing digestion of the protein obtained from ˜4 ul of the initial plasma sample (or ˜320 ug total protein). In FIG. 3, well 1 contains a stack 10 of multiple layers of a porous support suitable for immobilization of protein modifying reagents or enzymes.

In a further embodiment, a reagent-loaded membrane is provided as sheets, onto which protein sample can be pipetted. An area of sheet capable of completely imbibing the desired volume of sample is used: the sample volume in fact determines the area filled, and this area can be cut out or marked after sample is applied. For convenience, a soluble dye, inert with respect to the biomolecules-modifying activities of the membrane and its reagents, can be distributed uniformly across the membrane, for example by including it in the solution dried in the membrane when it is loaded with reagents or finally washed. The dye is soluble in sample, and thus dissolves in the applied sample volume, creating a colored ring that advances with the advancing sample liquid and marks the extent of sample coverage. If the sample is dried in the membrane, causing the disappearance of the membrane shading caused by imbibed liquid, this ring marks the region occupied by sample biomolecules. Bromophenol blue is an example of a suitable dye for protein applications.

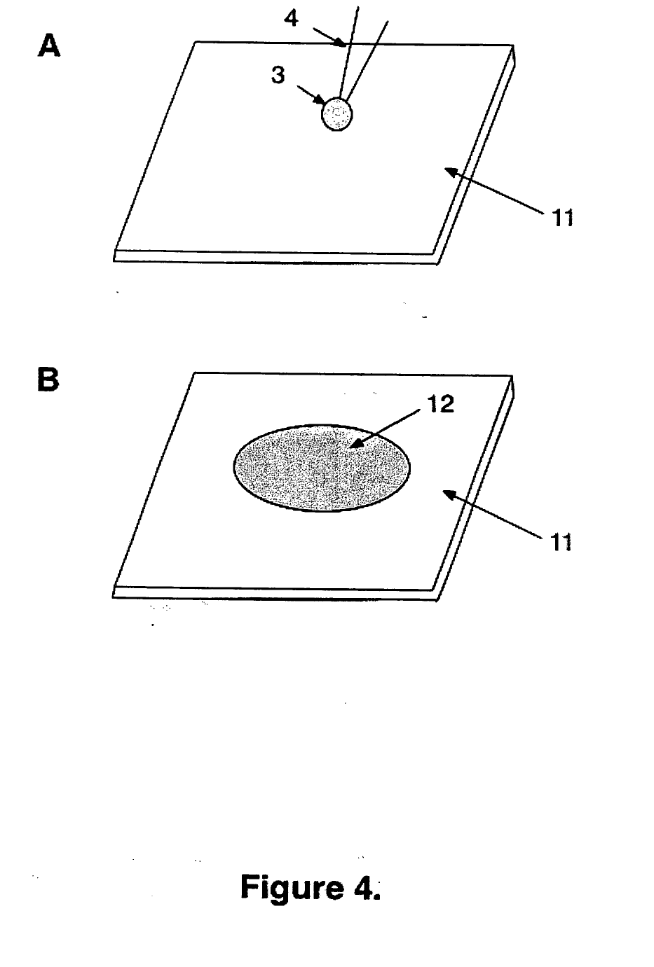

After digestion, peptides can be recovered by diffusion out of the membrane by placing it in a volume of buffer for this purpose, or else by filtration, in which case the membrane is placed on the frit of a vacuum filtration device. In FIG. 4A a volume of sample 3 is applied to porous support 11 by pipette 4. The sample is imbibed into membrane 11 and, when fully imbibed, occupies an area 12 of the membrane defined by the sample volume and the areal void volume of the membrane (FIG. 4B). Sample volume can be variable since the area 12 occupied by imbibed sample automatically adjusts to accommodate the entire applied sample. Area 12 can be excised for further processing.

Numerous alternative geometries and materials can be devised to effect these steps.

In a second group of embodiments mentioned above, a porous support is used to define a volume within which a known amount of a solid but dissolvable protein-modifying reagent is provided, but not immobilized. Here the porous support may be one of the many types used as a vehicle for reagents in lateral flow diagnostics (generally called a “conjugate release pad”).

In another preferred embodiment, a hydrophilic porous support (e.g., a nitrocellulose membrane with high capillary speed), preferably mounted on a non-porous plastic backing film, is filled by imbibition with a solution of protein denaturant (e.g., 6M GuHCl, 9M urea, or 1% sodium dodecyl sulfate) and then dried rapidly (or lyophilized) so as to produce finely dispersed dry particles of the denaturant uniformly distributed throughout the support.

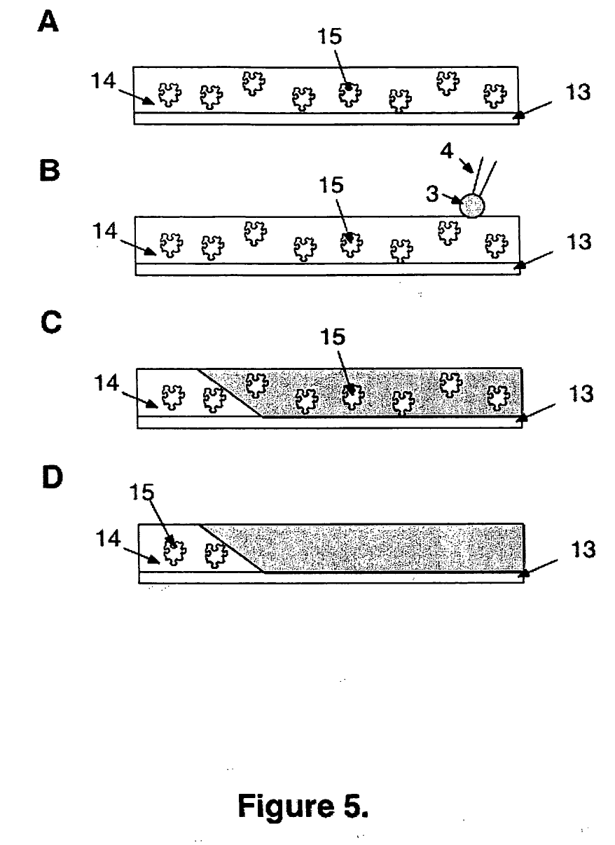

A protein-containing liquid sample (e.g., blood plasma) is then imbibed into the dry, denaturant-loaded membrane, where the denaturant dissolves into the sample at the originally applied concentration. Following a retention period, which may take place at room temperature or at an elevated temperature to speed protein denaturation, the denatured sample can be frozen, dried, or recovered from the membrane by diffusion into a volume of fluid to which the support is exposed. Since the included volume of the support defines the volume of denaturant solution initially taken up and dried, and this is substantially the same as the volume of protein-containing sample taken up and into which the denaturant redissolves, the final sample denaturant concentration is effectively the same as in the original denaturant solution. High capillary speed (or “lateral wicking speed”, the rate at which fluid spreads in the plane of the membranous support through capillary action) is desired in order that the sample is fully imbibed before the denaturant particles (or crystals) dissolve. These steps are shown in FIG. 5. In FIG. 5A, porous support 14, fixed to inert backing sheet 13, is loaded with the desired reagent concentration by imbibing a reagent solution and drying the membrane to produce reagent crystals 15. In FIG. 5B, sample 3 is applied to the surface of the membrane by pipette 4. In FIG. 5C, sample is imbibed into support 14, occupying an area determined by the sample volume, in this case less than the maximum available area. In FIG. 5D, reagent crystals 15 dissolve in the imbibed sample zone.

If the denaturant particles dissolve more rapidly than desired, leading to a higher concentration of denaturant at the advancing front of sample entering the membrane as compared to the sample remaining near the region of sample application (i.e., there is transport of initially-dissolved denaturant with the advancing sample front), the invention provides several optional approaches to produce a more constant denaturant concentration in a usable volume of membrane.

In a first option, dried denaturant particles in the membrane are coated by application of a layer of a slowly dissolving material of a type that does not interfere with subsequent sample analysis. Many such coating materials are known in the arts related to the formulation of timed-release pharmaceutical compositions, and include a variety of sugars, waxes, silicas, and organic polymers. In one optional approach, a coating material is selected that is slightly soluble in water and much more soluble in an organic solvent. After a denaturant such as GuHCl is dried in the membrane, a solution of coating material in organic solvent is sprayed onto the membrane, into which it is imbibed, and deposited as a coating on the membrane and on the crystals of dried denaturant and the organic solvent evaporates. When sample is applied to the membrane, it is imbibed to completion before the coating dissolves allowing contact between sample and denaturant. Under these circumstances the sample is stationary (fully imbibed) before denaturant dissolves, and thus the evenly distributed denaturant reaches equal concentration in all portions of the sample volume.

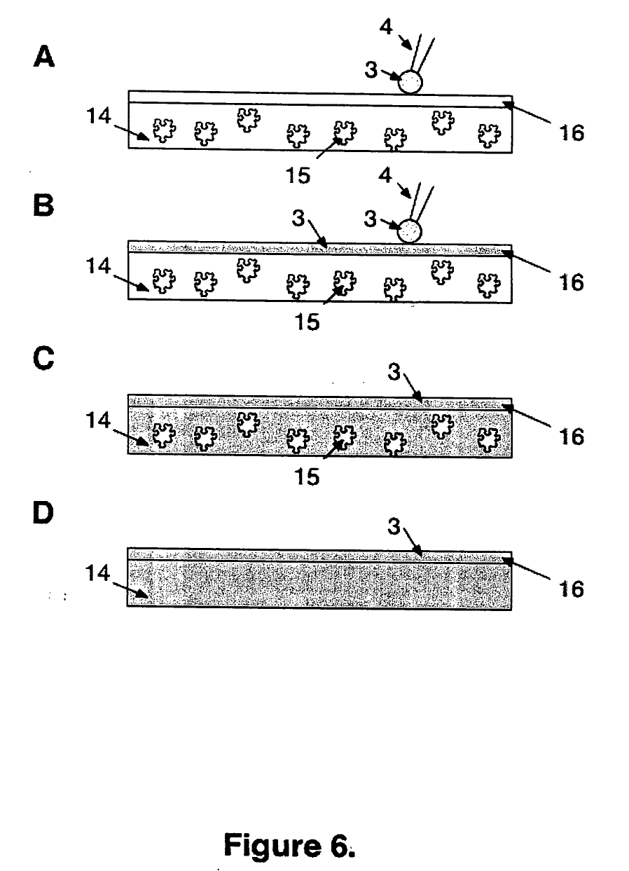

A second option to ensure equal concentrations of denaturant throughout the sample makes use of a two-layer membrane, in which a first layer, onto which sample is placed, has a very rapid lateral flow rate, and the second layer underlying the first layer contains the dried denaturant and has a lower later flow rate. In this case sample applied to the first layer spreads laterally over the entire area of the membrane, achieving a relatively equal loading of sample per unit area, and subsequently is imbibed by the second layer where the denaturant dissolves at substantially uniform concentration. As shown in FIG. 6A, an upper support 16 having very fast lateral wicking speed is layered above support 14 which contains crystals of dried reagent 15. In FIG. 6B, sample wicks laterally in support 16, spreading across the device. In FIG. 6C, after a short delay, sample is wicked more slowly into support 14, spreading over the crystals of reagent. In FIG. 6D, the reagent dissolves in the sample, yielding a uniform concentration.

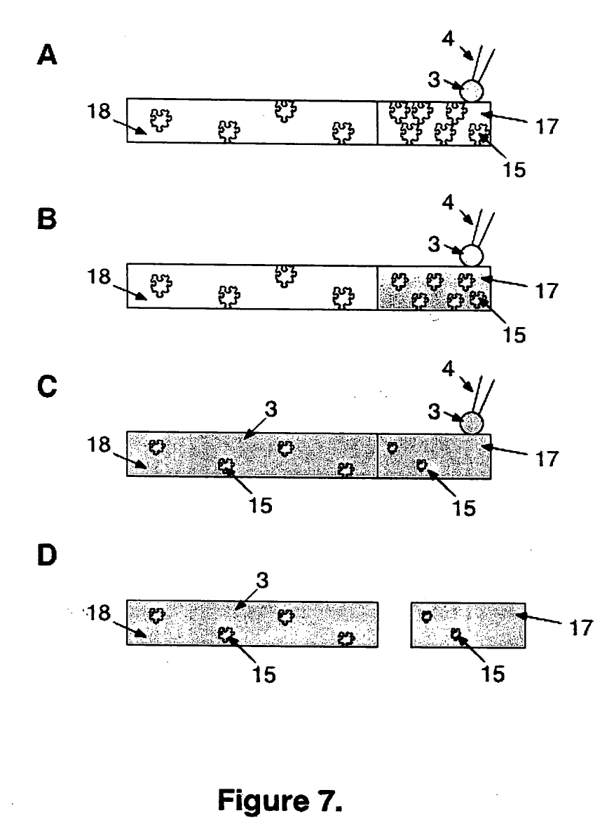

A third option provides two membrane zones connected by an edge (i.e., lying in the same plane): a first zone with which the sample is contacted and through which sample is imbibed into the membrane, and which contains a high loading of dried denaturant; and a second zone in which a lower loading of dried denaturant is present. As sample is applied to the first zone, denaturant rapidly dissolves and is transported laterally by capillary force into the second zone, where some additional denaturant continues to dissolve as sample becomes effectively saturated with denaturant. When both zones are filled and sample flow ceases, the first zone may be depleted of denaturant but the second zone is filled with sample saturated with denaturant. This approach is most effective when the objective is to saturate sample with denaturant, as is the case when using urea, for example. As shown in FIG. 7A, a support having a sample entry zone 17 and a sample recovery zone 18 are loaded with dried reagent 15, optionally with a higher reagent loading in the entry zone. Sample 3 is applied to the surface of the entry zone of the support 17 by pipette 4. In FIG. 7B, sample is imbibed into the entry zone, dissolving much of the reagent dried there. In FIG. 7C, sample imbibition continues through lateral flow of sample (now almost saturated with reagent) into the recovery zone 18, where some but not all of the remaining reagent is dissolved and the sample reaches saturation with reagent. In FIG. 7D, the recovery zone, which contains sample saturated with reagent, is separated from the loading zone, which is depleted of reagent.

After incubation for a period during which denaturation can occur (e.g., 15-120 minutes), a covering plastic film can be removed from the plasma- and reagent-filled membrane, allowing the denatured proteins to be dried in place, or removed by diffusion into a fluid volume (for example if the membrane on its substrate is inserted into a fluid containing tube.

A disulfide reductant can be included with the denaturant to improve its ability to unfold the proteins. One such reductant is dithiothreitol (although oxygen in the air will partially oxidize this material compromising its reducing power over time), while a preferable alternative is an agent such as tris [2-carboxyethyl] phosphine (TCEP).

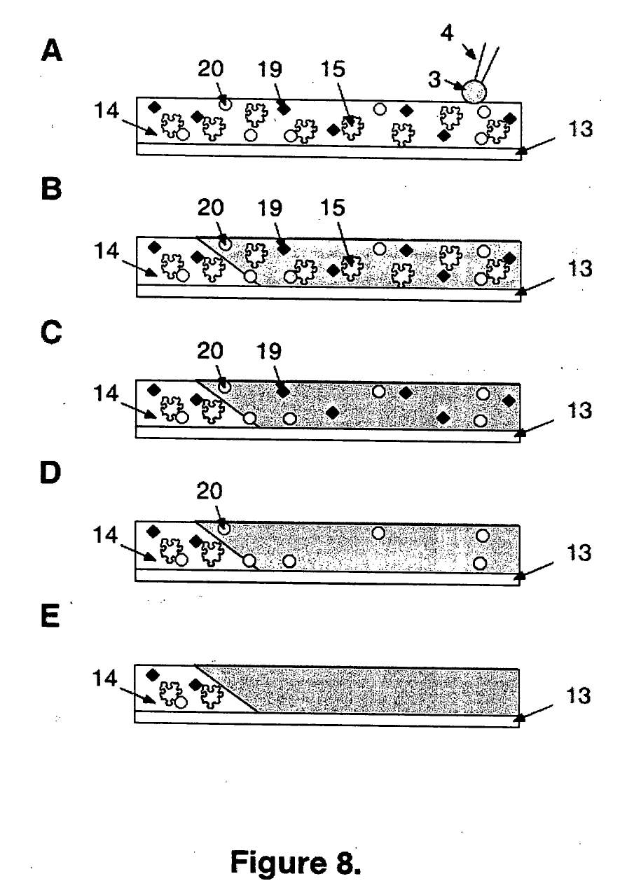

Multiple reagents may be placed in or on a porous support such as a membrane and some of these reagents are coated with different coatings so as to yield different dissolution times in sample. This embodiment is illustrated in FIG. 8. In FIG. 8A, porous support 14, fixed to inert backing foil 13, is loaded with three different reagents 15, 19, and 20, each of which is coated with a different coating formulation so as to delay its dissolution. Sample 3 is applied to the surface of the support by pipette 4. In FIG. 8B, sample is imbibed into the support, occupying an area determined by the applied volume. In FIG. 8C, particles of reagent 15 dissolve soon after imbibition. In FIG. 8D, particles of reagent 19 dissolved after an incubation period during which reagent 15 has carried out its function. In FIG. 8E, particles of reagent 20 dissolved after an incubation period during which reagent 19 has carried out its function.

For example, the process of cystine disulfide reduction and alkylation can be carried out in three steps separated by two incubations: 1) addition of reductant; 2) addition of alkylator; and 3) addition of more reductant to scavenge any excess remaining alkylator. A microparticulate preparation of alkylator (such as iodoacetamide; IAm) is coated with a coating material that delays dissolution of the alkylator in sample by approximately 15 minutes, while a second microparticulate preparation of reductant (such as dithiothreitol; DTT) is coated with a different or thicker coating material that delays dissolution in sample by approximately 30 minutes. The desired membrane is prepared by 1) imbibing a solution of denaturant and reductant, after which the membrane is dried and coated with a material that delays dissolution by 2 minutes (the time during which the membrane is filled with imbibed sample); and 2) suspensions of microparticulate alkylator (coated for a 15 min dissolution delay) and reductant (coated for a 30 min dissolution delay) are sprayed or otherwise applied onto the membrane at an even loading per area. In such a membrane, imbibed sample is first denatured and reduced for 15 minutes, after which time the alkylator microparticles dissolve and the cysteine residues are alkylated for 15 minutes in the dark, after which the final reductant particles dissolve, releasing reductant which scavenges any remaining unreacted alkylator. The result is to carry out a sequence of three sequential reactions in a single volume of sample with no manipulation other than application of sample to the membrane.

Because the region of the lower membrane containing the zone of dried denaturants can be covered over its upper and lower surfaces by opaque plastic films (only the upper surface of the blood filter membrane must be open for sample input), iodoacetamide in such a system would not be exposed to light, and hence remains relatively stable in a dried state.

A membrane support such as those prepared as using a hydrophilic support or multiple reagents placed in or on a porous support can be contacted with a blood filter membrane so as to allow whole blood to be applied as the original sample. Such membranes, specifically designed to trap blood cells (red cells, lymphocytes, etc) and platelets, while allowing the fluid plasma to pass, are well known in the art and commercially available from many sources. They include specific forms suitable for filtration through the plane of the membrane and also filtration in the plane of the membrane (i.e., lateral flow). These membranes are widely used in lateral-flow diagnostics test devices.

In this instance, a sample of whole blood is applied by pipette or needle or from the end of a pricked finger onto the exposed surface of a blood filter pad which wicks the sample while retaining cells and platelets on its surface and permitting plasma (the protein solution in which blood cells are suspended in the circulation) to flow through (either laterally in the plane of the membrane or else through the plane of the membrane to exit on the other side). The plasma thus prepared is drawn by capillary force into a reagent-loaded underlying (or laterally contacting) second membrane material in contact with the blood filter membrane and optionally mounted on a further underlying plastic substrate. When this second membrane has imbibed its full volume of plasma, flow ceases. The dried denaturants are subsequently dissolved in the plasma, denaturing the proteins and reducing disulfide bonds or further alkylated. After incubation for a period during which the desired protein modifications can occur, an optional impermeable covering plastic film can be removed from the plasma- and reagent-filled membrane, allowing the denatured proteins to be dried in place, or removed by diffusion into a fluid volume (for example if the membrane on its substrate is inserted into a fluid containing tube). An important feature of this embodiment is the spatial separation of the trapped blood cells from any biomolecules-modifying reagents: many of the latter, particularly protein denaturants, can cause cells to lyse and release cellular proteins into plasma. Thus the separated plasma is allowed to flow a short distance (typically 1 to 5 mm) through an empty imbibing membrane before encountering such reagents. This distance prevents the reagents from diffusing back to the trapped cells and lysing them during the period of device use.

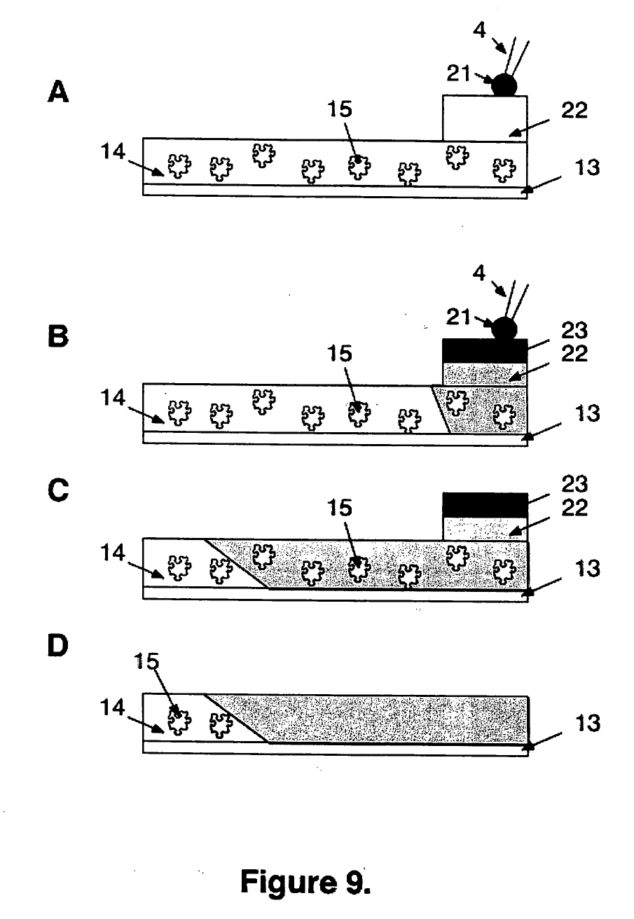

These steps are illustrated in FIG. 9. In FIG. 9A, porous support 14, fixed to inert backing sheet 13, is loaded with the desired reagent concentration by imbibing a reagent solution and drying the membrane to produce reagent crystals 15. A blood filter layer 22, capable of retaining blood cells and platelets while permitting flow of plasma, is layered above support 14, and whole blood 21 is applied to the surface of the blood filter 22 by pipette 4. In FIG. 9B and 9C, plasma is imbibed into support 14, occupying an area determined by the sample volume, while blood cells 23 remain in the filter. In FIG. 9D, reagent crystals 15 dissolve in the imbibed plasma zone.

Various combinations, such as those using hydrophilic membranes and multiple layers, can be employed in sequence to prepare a sample digest. First a sample (e.g., human plasma) is placed on a reagent-loaded membrane capable of carrying out the denaturation, reduction and alkylation of the sample proteins, prepared according to the fourth embodiment. The membrane forms the bottom of a 96-well filter plate, and is chosen for its low protein binding and its ability to imbibe sample. Following application of the imbibable volume of sample to the membrane (typically 3-6 ul), denaturation, reduction and alkylation occur during an incubation step (typically 1 hour). After the incubation, 50 ul of 100 mM ammonium bicarbonate in 40% propylene glycol, 60% water is added to each well and the denatured sample diluted by diffusion for a further hour. Next the denatured sample, now diluted to ˜55 ul, is pulled through the membrane by applied vacuum and collected in a receiver filter plate whose filter consists of 10 layers of trypsin-loaded PVDF (prepared according to the second preferred embodiment). The 55 ul diluted sample is completely imbibed, and cleaved to peptides by the immobilized trypsin during a 30 min incubation. Next 1 ml of 100 mM ammonium bicarbonate buffer is added to each well and the digested peptides pulled though the membrane by vacuum and into a receiver plate which in this case is an Empore® C18 filterplate (which has previously been wetted by methanol and equilibrated with the same buffer). The peptides bind to the C18 filter and are washed by application of several volumes of buffer pulled through by vacuum. Finally 200 ul of 50% acetonitrile in water is applied to each well and the digest peptides on the C18 filter plate are pulled through into a clean microwell plate by vacuum. The peptides are subsequently dried and redissolved in 20 ul of a solvent appropriate for injection into the LC-MS/MS analytical system (typically 0.2% trifluoroacetic acid in water). In each of the three stages at which vacuum is used, centrifugal force could be used instead to move liquid through the filter plate membranes. It is understood that for each of these fluid transfers, the source plate containing sample components and the receiver plate are assembled source above receiver and inserted into an appropriate device (either for vacuum filtration or centrifugation).

### EXAMPLE 1

MSIPN4510 96-well MultiScreenHTS Plates with hydrophobic Immobilon-P PVDF membrane are purchased from Millipore and used as support. Trypsin is purchased from Sigma. A vacuum filtration device (Millipore MultiScreen™ Vacuum Manifold 96-well) is used to pull liquids through the filters into the wells of a receiver 96-well plate below the filter plate using a vacuum of 9″ Hg.

The PVDF membranes are first wetted by loading each well with 0.05 ml of 100% methanol, followed immediately by 0.25 ml of 0.1M ammonium bicarbonate buffer pH 8.0, which is pulled through the membrane by vacuum. Three additional aliquots of 0.25 ml of 0.1M ammonium bicarbonate buffer are loaded and pulled through the membranes in each well. Next the membranes are trypsin-coated by application of 100 ul of 2 mg/ml trypsin in ammonium bicarbonate buffer with 20 mM calcium chloride, pH 8.0, which is pulled slowly through the membranes by vacuum. Lastly, the membranes are washed by passage through each well of five volumes (0.3 ml) of 50 mM acetic acid. In all of the preceding steps, vacuum is carefully controlled so as to avoid drying of the membrane before the next volume of liquid is applied. Following the last wash, the vacuum is continued to draw air through the membranes until they are dry. The matrix of the porous membrane of each well is thus coated with approximately 35 ug trypsin, yielding a very high local enzyme concentration (35 ug/6 ul=˜6 mg/ml). Once dried, the membrane is stable for long-term storage.

A sample of human plasma is prepared for digestion by denaturation, reduction and alkylation. Briefly, 10 ul of plasma is subjected to denaturation by addition of 20 ul of 8M guanidine HCl in 100 mM ammonium bicarbonate pH 8.0 (yielding a GuHCl concentration of 5.3M) and 5 ul of 200 mM dithiothreitol (DTT) in water, and incubated for 30 minutes at 50° C. After the sample has cooled to room temperature, cysteine residues are then alkylated by addition of 5 ul of 400 mM iodoacetamide (IAm) in water, followed by incubation at room temperature in the dark for 15 minutes. Excess IAm is consumed by a further addition of 10 ul 200 mM DTT in water, followed by incubation for 5 minutes at room temperature. Finally, the GuHCl is further diluted (to 1.0M, at which trypsin can function) by addition of 110 ul of 50% propylene glycol in 100 mM ammonium bicarbonate pH 8.0. The final sample contains approximately 5.0 mg/ml plasma protein (a 16-fold dilution from plasma), 1.0M GuHCl, and 34.4% propylene glycol.

To initiate digestion, a 6 ul aliquot of the denatured plasma protein sample (containing ˜30 ug protein) is applied to the center of the trypsin-loaded PVDF membrane at the bottom of a well. While a conventional aqueous sample would not wet the PVDF (even after it receives its coating of trypsin), a sample containing ˜35% propylene glycol does wet, and is imbibed into the membrane over a period of approximately 45 sec. The volume (6 ul) is chosen to be slightly less than the measured inclusion volume of the membrane forming the well bottom of the filter plate. Thus the sample is entirely imbibed into the included volume of the membrane, and virtually no free liquid remains on the membrane surface.

At this high enzyme:substrate ratio (close to 1:1), the digestion proceeds quickly at room temperature. Thus after a 1-10 min incubation at room temperature, 250 ul of 100 mM ammonium bicarbonate pH 8.0 is introduced into the well, and the filterplate is placed over a receiver plate in the vacuum filtration device. The buffer is then drawn through the PVDF by applied vacuum, and the flow-through liquid, containing the peptides, is collected in the receiver plate. After this transfer, the receiver plate is retrieved and shaken to mix the peptides and wash solution.

In the final stage of digest cleanup, the peptide samples are introduced into wells of a 3M Empore© C18 filter plate whose membranes have previously been wetted with methanol and equilibrated with 100 mM ammonium bicarbonate pH 8.0 by washing. The vacuum device is then reassembled with the Empore filterplate as the top component, and the peptide solution drawn through it by vacuum. The flowthrough is discarded (since it is depleted of peptides, which are captured on the C18 material of the Empore plate). The Empore plate is successively washed with three loads of 500 ul of 100 mM ammonium bicarbonate pH 8.0, and dried briefly by passage of air through the filters. Finally the Empore plate is placed over a fresh receiver plate, and a peptide-eluting solution (50% acetonitrile, 0.1% TFA in water) is applied to each well and vacuum used to draw this solution through the C18 bed, eluting the peptides into the final receiver plate. This final plate is then subjected to evaporation in a SpeedVac so as to dry the peptides down. These peptide samples represent the input sample to the LC-MS/MS. analytical platform, and are redissolved into an aqueous solvent for application to such a system.

### EXAMPLE 2

In this example, plasma is obtained from a sample of whole blood obtained by fingerprick, the plasma proteins denatured stabilized for storage by drying. A wicking membrane (Predator, provided on a thin impermeable polyester support) and a blood filter membrane (BTS Highly Asymmetric Membrane BTS-100) are purchased from Pall Corporation.

A strip of Predator membrane 1 cm×10 cm is prepared. A solution of 6M guanidine hydrochloride (protein denaturant) in water, containing a trace of bromophenol blue marker dye (enough to impart a blue color), is loaded onto one end of the strip and allowed to wick over a total length of 7 cm from the application end (leaving 3 cm at the other end dry). Any excess liquid on the membrane surface is removed to prevent further wicking. The wetted portion of the membrane is rapidly dried by application of hot air to the surface (using, for example, a common hairdryer).

This reagent-loaded strip is placed on a horizontal surface. A piece of BTS blood filter membrane is cut to 1 cm×1 cm and placed (with the low-porosity side down) on top of the Predator strip at a position 1 cm removed from the edge of the denaturant zone (and thus covering the section of the strip between 8 cm and 9 cm from the end where denaturant was originally applied). The BTS square is aligned with the edges of the Predator strip so that it does not extend over the strip at the sides, and is fully supported by the strip underneath.

The resulting device is used by executing two steps. First a small volume of whole blood, obtained using a fingerprick device (such as the Becton Dickinson Lancet Device) is applied to the center of the BTS pad on top of the Predator strip, ensuring that the blood does not flow over the edges of the BTS material. A volume of 10-20 ul of blood is appropriate. After an interval of 1 minute, during which plasma filters through the BTS membrane and into the region of the Predator strip underneath it, a volume of 50 ul of Hank's Balanced Salt Solution (HBSS; Sigma-Aldrich Chemicals) is applied to the dry end of the strip (the 1 cm exposed region adjacent to the BTS pad and opposite the end where the denaturant was originally applied). This HBSS then wicks under the BTS pad and transports plasma into the dry membrane loaded with denaturant. Additional HBSS is loaded at the HBSS application point until the liquid front reaches the other end of the strip.

At this point the BTS pad is preferably removed and discarded (unless cellular components are required, in which case the pad and its trapped cells can be retained for further processing). The denaturant-containing zone, now including the plasma proteins in 6M GuHCl, is dried using a hairdryer and placed in moisture-impermeable plastic bag with a MiniPax® desiccant packet and a Freshpax oxygen absorber packet (Multisorb Technologies, Inc.) to maintain dryness and reduce oxygen capable of oxidizing proteins. The blood-sample-derived plasma proteins are now in a dry/denatured state in which they are stable during transportation to a site of analysis by US Mail or by courier services. At the site of analysis (or sample reconstitution), the strip is removed from the plastic bag, and the proteins extracted from the strip by soaking the protein-containing end in a small trough containing 200 ul of water. This resulting recovered protein solution is then suitable for digestion using a proteolytic enzyme such as trypsin and subsequent analysis by liquid chromatography/mass spectrometry in order to quantitate specific proteins as biomarkers.

What I claim is:

1. A process for treatment of biomolecules comprising the steps of:
1) preparing a porous support incorporating one or more biomolecule-modifying reagents,
2) applying a biomolecule-containing sample solution to said support wherein the volume of said sample is equal to or less than the imbibition capacity of said support so that the sample is completely imbibed into said support,
3) allowing the product of step 2 sufficient time for said biomolecule-modifying reagent to interact with the biomolecule of interest in said sample, then
4) recovering biomolecules from said support.

2. The process of claim 1 wherein, after step 3, the product of step 3 is stored before the recovery step.

3. The process of claim 2 wherein the product of step 3 is stored as a dried product before the recovery step.

4. The process of claim 1 wherein said sample is imbibed into said support by capillary action.

5. The process of claim 1 wherein said support contains at least one of particles, a membrane, a porous film, a woven or non-woven fabric, a sintered aggregate of particles, a foam or a monlithic support.

6. The process of claim 1 wherein said support comprises at least two phases.

7. A device for use in analysis of at least one biomolecule comprising a walled container with an exit opening spanned by a support, said support having therein at least one biomolecule modifying reagent, said support having imbibed therein a cell-free biomolecule-containing sample wherein the volume of the total sample applied to said support in said walled container is less than or equal to the imbibition capacity of said support.

8. A device for use in analysis of a biomolecule-containing material comprising a filter impermiable to cells in contact with a support within which there is incorporated in said support at least one biomolecule-modifying agents.

9. A device of claim 8 wherein ay least one biomolecule-modifying agent is a digesting or denaturing agent.

10. The device of claim 7 wherein the support comprises at least one of the biomolecule-modifying reagents on support materials chosen from particles, membranes, porous films, woven or non-woven fabric, sintered aggregate of particles, foam or monolithic support.

11. The process of claim 1 wherein, in step 2, the biomolecule-containing sample is imbibed as said sample flows within the plane of said support.

12. The process of claim 1 wherein at least one of said reagents is formulated, coated or encapsulated to delay its action on said biomolecules relative to at least one other said reagent.

13. The process of claim 1 wherein said support incorporates at least one reagent chosen from denaturants, sulfhydral reducing agents, thiol-reactive compounds, proteolytic enzymes, protease inhibitors, detergents and salts.

14. The process of claim 1 wherein at least one said biomolecule-modifying reagent is bound to said support co-valently.

15. The process of claim 1 wherein at least one said biomolecule-modifying reagent is bound to said support non-covanlently

16. The process of claim 14 wherein at least one said biomolecule-modifying reagent is cross-linked after binding to said support

17. The device of claim 7 wherein one biomolecule-modifying reagent is an enzyme wherein said enzyme is bound to said support.

18. The process of claim 1 wherein, in step 1, at least one biomolecule-modifying reagent incorporated in said support is not bound to said support.

19. The process of claim 1 wherein, before step 2 a wetting agent is added to said sample before inbibition of step 3.

20. The process of claim 1 wherein at least one membrane is interposed between the sample and said support incorporating one or more biomolecule-modifying reagents.
